(19) United States
(12) Patent Application Publication
(10) Pub. No.: US 2023/0280482 A1
(43) Pub. Date: Sep. 7, 2023

(54) IMAGING SYSTEMS

(71) Applicant: SHENZHEN XPECTVISION TECHNOLOGY CO., LTD., Shenzhen (CN)

(72) Inventors: Yurun LIU, Shenzhen (CN); Peiyan CAO, Shenzhen (CN)

(21) Appl. No.: 18/195,992

(22) Filed: May 11, 2023

Related U.S. Application Data
(63) Continuation of application No. PCT/CN2020/131469, filed on Nov. 25, 2020.

(51) Int. Cl. G01T 1/24; H04N 5/32

(52) U.S. Cl. CPC G01T 1/247; H04N 5/32

(57) ABSTRACT

Disclosed herein is a method, comprising: scanning a scene for a first scan in a scanning direction with M detector blocks (detector blocks (i), i=1, . . . , M), wherein the M detector blocks are physically arranged in the order of the detector blocks (1), (2), . . . , (M) in the scanning direction during the first scan, M being an integer greater than 1; and after the first scan, scanning the scene for a second scan in the scanning direction with the M detector blocks, wherein the M detector blocks are physically arranged in the order of the detector blocks (M), (1), (2), . . . , (M−1) in the scanning direction during the second scan.

### TECHNICAL FIELD

The disclosure herein relates to imaging systems.

### BACKGROUND

A radiation detector is a device that measures a property of a radiation. Examples of the property may include a spatial distribution of the intensity, phase, and polarization of the radiation. The radiation may be one that has interacted with an object. For example, the radiation measured by the radiation detector may be a radiation that has penetrated the object. The radiation may be an electromagnetic radiation such as infrared light, visible light, ultraviolet light, X-ray or γ-ray. The radiation may be of other types such as α-rays and β-rays. An imaging system may include multiple radiation detectors.

### SUMMARY

Disclosed herein is a method, comprising: scanning a scene for a first scan in a scanning direction with M detector blocks (detector blocks (i), i=1, . . . , M), wherein the M detector blocks are physically arranged in the order of the detector blocks (1), (2), . . . , (M) in the scanning direction during the first scan, M being an integer greater than 1; and after the first scan, scanning the scene for a second scan in the scanning direction with the M detector blocks, wherein the M detector blocks are physically arranged in the order of the detector blocks (M), (1), (2), . . . , (M−1) in the scanning direction during the second scan.

In an aspect, the method further comprises, after the second scan, scanning the scene for a third scan in the scanning direction with the M detector blocks, wherein the M detector blocks are physically arranged in the order of the detector blocks (M−1), (M), (1), (2), . . . , (M−2) in the scanning direction during the third scan, and wherein M>2.

In an aspect, each detector block of the M detector blocks comprises a radiation detector.

In an aspect, during each scan of the first scan and the second scan, the M detector blocks are stationary with respect to each other.

In an aspect, during each scan of the first scan and the second scan, the M detector blocks are distributed evenly in the scanning direction.

In an aspect, said scanning for the first scan comprises capturing first H partial images while the M detector blocks are moving, H being an integer greater than 1, and said scanning for the second scan comprises capturing second H partial images while the M detector blocks are moving.

In an aspect, the first H partial images are stitchable together, and the second H partial images are stitchable together.

In an aspect, the method further comprises: stitching the first H partial images to form an image; and stitching the second H partial images to form an image.

In an aspect, the method further comprises: after the first scan and before the second scan, moving the detector block (M) along a path, wherein at a time point after the first scan and before the second scan, a point on the path is in shadows of the other detector blocks of the M detector blocks with respect to radiation used for said first scan and said second scan.

In an aspect, the detector block (M) flips twice while being moved along the path after the first scan and before the second scan.

In an aspect, each detector block of the M detector blocks comprises multiple radiation detectors, the multiple radiation detectors of said each detector block are stationary with respect to each other, and projections of active areas of the multiple radiation detectors of said each detector block on a plane perpendicular to radiation used in the first and second scans collectively form a single region on the plane.

Disclosed herein is an imaging system, comprising M detector blocks (detector blocks (i), i=1, . . . , M), with M being an integer greater than 1, wherein the M detector blocks are configured to scan a scene for a first scan in a scanning direction, wherein the M detector blocks are physically arranged in the order of the detector blocks (1), (2), . . . , (M) in the scanning direction during the first scan, and wherein the M detector blocks are configured to scan the scene for a second scan after the first scan, in the scanning direction, wherein the M detector blocks are physically arranged in the order of the detector blocks (M), (1), (2), . . . , (M−1) in the scanning direction during the second scan.

In an aspect, the M detector blocks are configured to scan the scene for a third scan after the second scan, in the scanning direction, the M detector blocks are physically arranged in the order of the detector blocks (M−1), (M), (1), (2), . . . , (M−2) in the scanning direction during the third scan, and M>2.

In an aspect, each detector block of the M detector blocks comprises a radiation detector.

In an aspect, during each scan of the first scan and the second scan, the M detector blocks are stationary with respect to each other.

In an aspect, during each scan of the first scan and the second scan, the M detector blocks are distributed evenly in the scanning direction.

In an aspect, during the first scan, the M detector blocks are configured to capture first H partial images while the M detector blocks are moving, H being an integer greater than 1, and during the second scan, the M detector blocks are configured to capture second H partial images while the M detector blocks are moving.

In an aspect, the first H partial images are stitchable together, and the second H partial images are stitchable together.

In an aspect, the imaging system is configured to stitch the first H partial images to form an image, and the imaging system is configured to stitch the second H partial images to form an image.

In an aspect, after the first scan and before the second scan, the imaging system is configured to move the detector block (M) along a path, and at a time point after the first scan and before the second scan, a point on the path is in shadows of the other detector blocks of the M detector blocks with respect to radiation used for said first scan and said second scan.

In an aspect, the imaging system is configured to flip the detector block (M) twice while the detector block (M) is moved along the path after the first scan and before the second scan.

In an aspect, each detector block of the M detector blocks comprises multiple radiation detectors, the multiple radiation detectors of said each detector block are stationary with respect to each other, and projections of active areas of the multiple radiation detectors of said each detector block on a plane perpendicular to radiation used in the first and second scans collectively form a single region on the plane.

### BRIEF DESCRIPTION OF FIGURES

FIG. 1 schematically shows a radiation detector, according to an embodiment.

FIG. 2A schematically shows a simplified cross-sectional view of the radiation detector.

FIG. 2B schematically shows a detailed cross-sectional view of the radiation detector.

FIG. 2C schematically shows an alternative detailed cross-sectional view of the radiation detector.

FIG. 3 schematically shows a top view of a package including the radiation detector and a printed circuit board (PCB).

FIG. 4 schematically shows a cross-sectional view of a detector module, where a plurality of the packages of FIG. 3 are mounted to a system PCB, according to an embodiment.

FIG. 5A-FIG. 5D schematically show top views of the detector module in operation, according to an embodiment.

FIG. 6A-FIG. 6E schematically illustrate an operation of an imaging system, according to an embodiment.

FIG. 7 shows a flowchart summarizing and generalizing an operation of the imaging system, according to an embodiment.

FIG. 8A-FIG. 8C schematically illustrate an operation of the imaging system during a reset, according to an embodiment.

FIG. 9A-FIG. 9B schematically illustrate a detector block, according to an embodiment.

### DETAILED DESCRIPTION

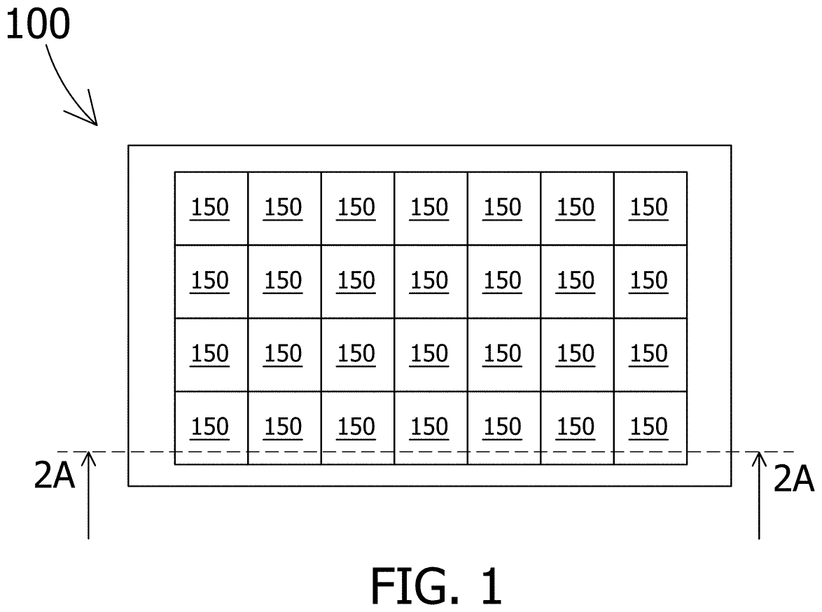

FIG. 1 schematically shows a radiation detector 100, as an example. The radiation detector 100 may include an array of pixels 150 (also referred to as sensing elements 150). The array may be a rectangular array (as shown in FIG. 1), a honeycomb array, a hexagonal array or any other suitable array. The array of pixels 150 in the example of FIG. 1 has 28 pixels 150 arranged in 4 rows and 7 columns; in general, the array of pixels 150 may have any number of pixels 150 arranged in any way.

A radiation may include particles such as photons (electromagnetic waves) and subatomic particles (e.g., neutrons, protons, electrons, alpha particles, etc.) Each pixel 150 may be configured to detect radiation incident thereon and may be configured to measure a characteristic (e.g., the energy of the particles, the wavelength, and the frequency) of the incident radiation. The measurement results for the pixels 150 of the radiation detector 100 constitute an image of the radiation incident on the pixels. It may be said that the image is of an object or a scene which the incident radiation come from.

Each pixel 150 may be configured to count numbers of particles of radiation incident thereon whose energy falls in a plurality of bins of energy, within a period of time. All the pixels 150 may be configured to count the numbers of particles of radiation incident thereon within a plurality of bins of energy within the same period of time. When the incident particles of radiation have similar energy, the pixels 150 may be simply configured to count numbers of particles of radiation incident thereon within a period of time, without measuring the energy of the individual particles of radiation.

Each pixel 150 may have its own analog-to-digital converter (ADC) configured to digitize an analog signal representing the energy of an incident particle of radiation into a digital signal, or to digitize an analog signal representing the total energy of a plurality of incident particles of radiation into a digital signal. The pixels 150 may be configured to operate in parallel. For example, when one pixel 150 measures an incident particle of radiation, another pixel 150 may be waiting for a particle of radiation to arrive. The pixels 150 may not have to be individually addressable.

The radiation detector 100 described here may have applications such as in an X-ray telescope, X-ray mammography, industrial X-ray defect detection, X-ray microscopy or microradiography, X-ray casting inspection, X-ray non-destructive testing, X-ray weld inspection, X-ray digital subtraction angiography, etc. It may be suitable to use this radiation detector 100 in place of a photographic plate, a photographic film, a PSP plate, an X-ray image intensifier, a scintillator, or another semiconductor X-ray detector.

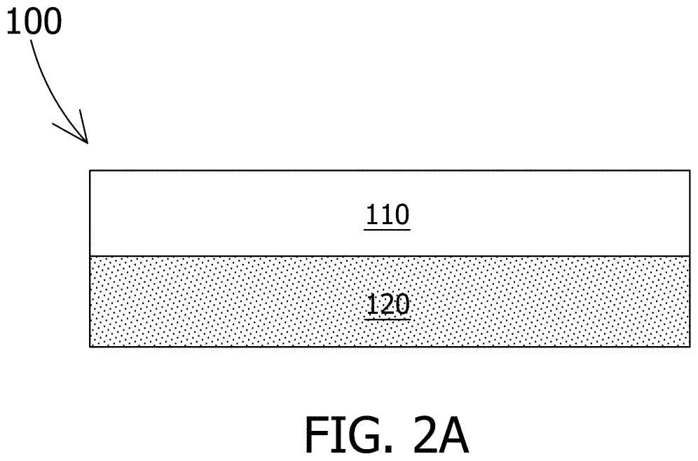

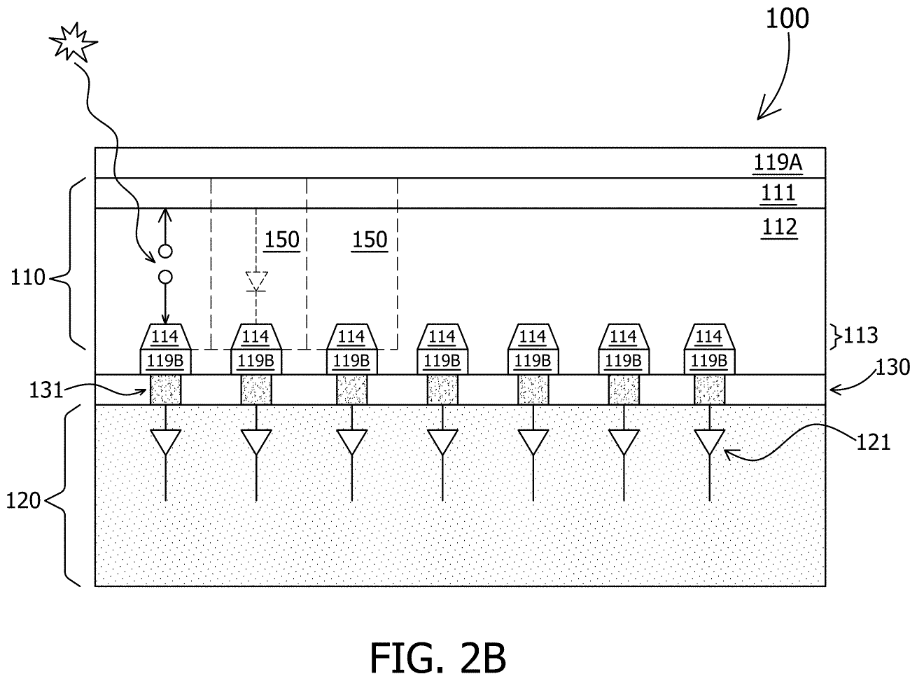

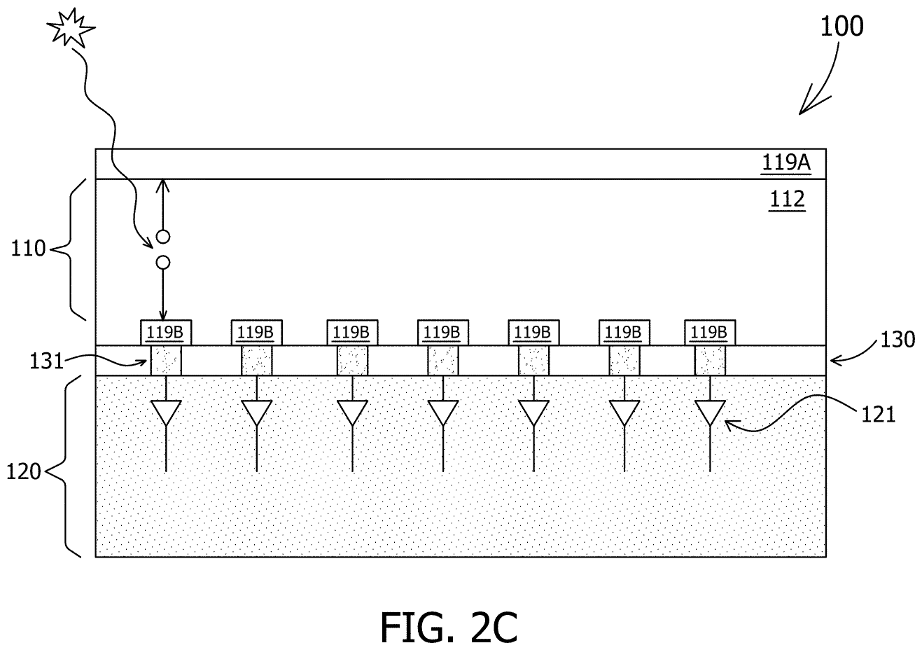

FIG. 2A schematically shows a simplified cross-sectional view of the radiation detector 100 of FIG. 1 along a line 2A-2A, according to an embodiment. More specifically, the radiation detector 100 may include a radiation absorption layer 110 and an electronics layer 120 (e.g., an ASIC) for processing or analyzing electrical signals which incident radiation generates in the radiation absorption layer 110. The radiation detector 100 may or may not include a scintillator (not shown). The radiation absorption layer 110 may comprise a semiconductor material such as silicon, germanium, GaAs, CdTe, CdZnTe, or a combination thereof. The semiconductor material may have a high mass attenuation coefficient for the radiation of interest.

FIG. 2B schematically shows a detailed cross-sectional view of the radiation detector 100 of FIG. 1 along the line 2A-2A, as an example. More specifically, the radiation absorption layer 110 may include one or more diodes (e.g., p-i-n or p-n) formed by a first doped region 111 and one or more discrete regions 114 of a second doped region 113. The second doped region 113 may be separated from the first doped region 111 by an optional intrinsic region 112. The discrete regions 114 are separated from one another by the first doped region 111 or the intrinsic region 112. The first doped region 111 and the second doped region 113 have opposite types of doping (e.g., region 111 is p-type and region 113 is n-type, or region 111 is n-type and region 113 is p-type). In the example of FIG. 2B, each of the discrete regions 114 of the second doped region 113 forms a diode with the first doped region 111 and the optional intrinsic region 112. Namely, in the example in FIG. 2B, the radiation absorption layer 110 has a plurality of diodes (more specifically, FIG. 2B shows 7 diodes corresponding to 7 pixels 150 of one row in the array of FIG. 1, of which only 2 pixels 150 are labeled in FIG. 2B for simplicity). The plurality of diodes may have an electrode 119A as a shared (common) electrode. The first doped region 111 may also have discrete portions.

The electronics layer 120 may include an electronic system 121 suitable for processing or interpreting signals generated by the radiation incident on the radiation absorption layer 110. The electronic system 121 may include an analog circuitry such as a filter network, amplifiers, integrators, and comparators, or a digital circuitry such as a microprocessor, and memory. The electronic system 121 may include one or more ADCs. The electronic system 121 may include components shared by the pixels 150 or components dedicated to a single pixel 150. For example, the electronic system 121 may include an amplifier dedicated to each pixel 150 and a microprocessor shared among all the pixels 150. The electronic system 121 may be electrically connected to the pixels 150 by vias 131. Space among the vias may be filled with a filler material 130, which may increase the mechanical stability of the connection of the electronics layer 120 to the radiation absorption layer 110. Other bonding techniques are possible to connect the electronic system 121 to the pixels 150 without using the vias 131.

When radiation from the radiation source (not shown) hits the radiation absorption layer 110 including diodes, particles of the radiation may be absorbed and generate one or more charge carriers (e.g., electrons, holes) by a number of mechanisms. The charge carriers may drift to the electrodes of one of the diodes under an electric field. The field may be an external electric field. The electrical contact 119B may include discrete portions each of which is in electrical contact with the discrete regions 114. The term “electrical contact” may be used interchangeably with the word “electrode.” In an embodiment, the charge carriers may drift in directions such that the charge carriers generated by a single particle of the radiation are not substantially shared by two different discrete regions 114 (“not substantially shared” here means less than 2%, less than 0.5%, less than 0.1%, or less than 0.01% of these charge carriers flow to a different one of the discrete regions 114 than the rest of the charge carriers). Charge carriers generated by a particle of the radiation incident around the footprint of one of these discrete regions 114 are not substantially shared with another of these discrete regions 114. A pixel 150 associated with a discrete region 114 may be a space around the discrete region 114 in which substantially all (more than 98%, more than 99.5%, more than 99.9%, or more than 99.99% of) charge carriers generated by a particle of the radiation incident therein flow to the discrete region 114. Namely, less than 2%, less than 1%, less than 0.1%, or less than 0.01% of these charge carriers flow beyond the pixel 150.

FIG. 2C schematically shows an alternative detailed cross-sectional view of the radiation detector 100 of FIG. 1 along the line 2A-2A, according to an embodiment. More specifically, the radiation absorption layer 110 may include a resistor of a semiconductor material such as silicon, germanium, GaAs, CdTe, CdZnTe, or a combination thereof, but does not include a diode. The semiconductor material may have a high mass attenuation coefficient for the radiation of interest. In an embodiment, the electronics layer 120 of FIG. 2C may be similar to the electronics layer 120 of FIG. 2B in terms of structure and function.

When the radiation hits the radiation absorption layer 110 including the resistor but not diodes, it may be absorbed and generate one or more charge carriers by a number of mechanisms. A particle of the radiation may generate 10 to 100,000 charge carriers. The charge carriers may drift to the electrical contacts 119A and 119B under an electric field. The electric field may be an external electric field. The electrical contact 119B includes discrete portions. In an embodiment, the charge carriers may drift in directions such that the charge carriers generated by a single particle of the radiation are not substantially shared by two different discrete portions of the electrical contact 119B (“not substantially shared” here means less than 2%, less than 0.5%, less than 0.1%, or less than 0.01% of these charge carriers flow to a different one of the discrete portions than the rest of the charge carriers). Charge carriers generated by a particle of the radiation incident around the footprint of one of these discrete portions of the electrical contact 119B are not substantially shared with another of these discrete portions of the electrical contact 119B. A pixel 150 associated with a discrete portion of the electrical contact 119B may be a space around the discrete portion in which substantially all (more than 98%, more than 99.5%, more than 99.9% or more than 99.99% of) charge carriers generated by a particle of the radiation incident therein flow to the discrete portion of the electrical contact 119B. Namely, less than 2%, less than 0.5%, less than 0.1%, or less than 0.01% of these charge carriers flow beyond the pixel associated with the one discrete portion of the electrical contact 119B.

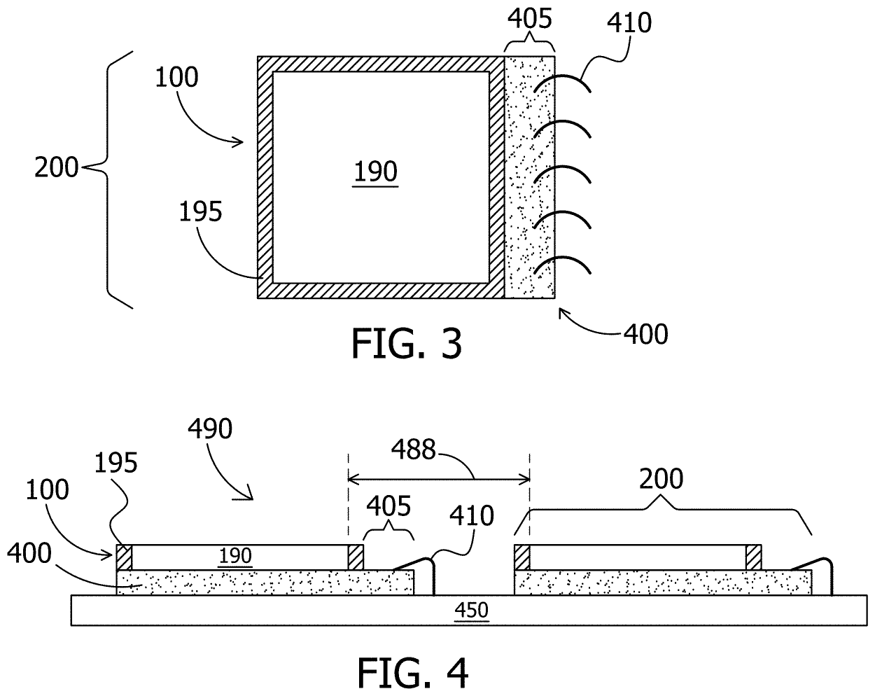

FIG. 3 schematically shows a top view of a package 200 including the radiation detector 100 and a printed circuit board (PCB) 400. The term “PCB” as used herein is not limited to a particular material. For example, a PCB may comprise a semiconductor. The radiation detector 100 may be mounted to the PCB 400. The wiring between the detector 100 and the PCB 400 is not shown for the sake of clarity. The PCB 400 may have one or more radiation detectors 100. The PCB 400 may have an area 405 not covered by the radiation detector 100 (e.g., for accommodating bonding wires 410). The radiation detector 100 may have an active area 190, which is where the pixels 150 (FIG. 1) are located. The radiation detector 100 may have a perimeter zone 195 near the edges of the radiation detector 100. The perimeter zone 195 has no pixels 150, and the radiation detector 100 does not detect particles of radiation incident on the perimeter zone 195.

FIG. 4 schematically shows a cross-sectional view of a detector module 490, according to an embodiment. The detector module 490 may include one or a plurality of the packages 200 of FIG. 3 mounted to a system PCB 450. FIG. 4 shows only 2 packages 200 as an example. The electrical connection between the PCBs 400 and the system PCB 450 may be made by bonding wires 410. In order to accommodate the bonding wires 410 on the PCB 400, the PCB 400 may have the area 405 not covered by the detector 100. In order to accommodate the bonding wires 410 on the system PCB 450, the packages 200 may have gaps in between. The gaps may be approximately 1 mm or more. Particles of radiation incident on the perimeter zones 195, on the area 405, or on the gaps cannot be detected by the packages 200 on the system PCB 450.

A dead zone of a radiation detector (e.g., the radiation detector 100) is the area of the radiation-receiving surface of the radiation detector, in which incident particles of radiation cannot be detected by the radiation detector. A dead zone of a package (e.g., package 200) is the area of the radiation-receiving surface of the package, in which incident particles of radiation cannot be detected by the detector or detectors in the package. In this example shown in FIG. 3 and FIG. 4, the dead zone of the package 200 includes the perimeter zones 195 and the area 405. A dead zone (e.g., 488) of a detector module (e.g., detector module 490) with a group of packages (e.g., packages mounted on the same PCB, packages arranged in the same layer) includes the combination of the dead zones of the packages in the group and the gaps among the packages.

In an embodiment, the detector module 490 including the radiation detectors 100 may have the dead zone 488 incapable of detecting incident radiation. However, in an embodiment, the detector module 490 with physically separate active areas 190 may capture partial images of incident radiation. In an embodiment, these captured partial images are such that they can be stitched by the detector module 490 to form a single image of incident radiation. In an embodiment, these captured partial images may be stitched to form a single image.

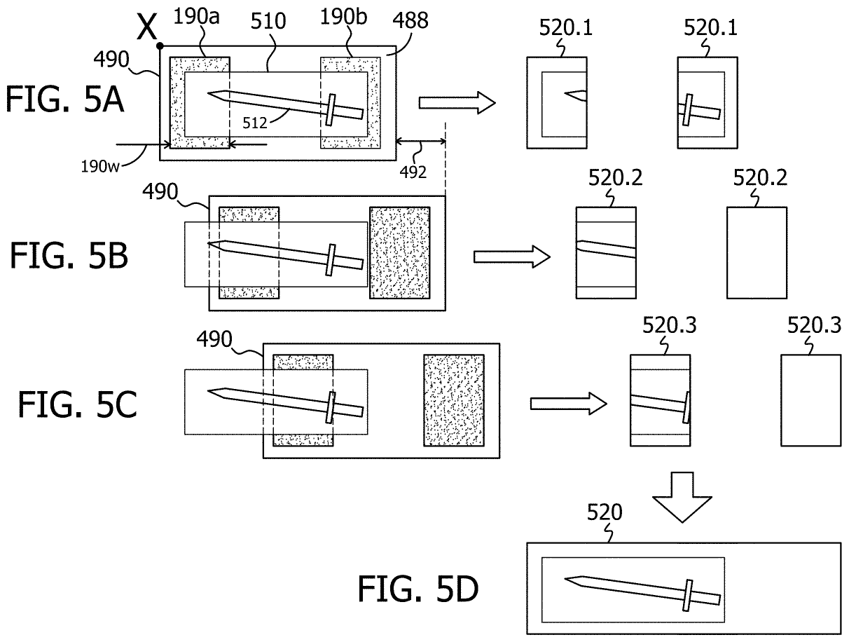

FIG. 5A-FIG. 5D schematically show top views of the detector module 490 in operation, according to an embodiment. In an embodiment, the detector module 490 may comprise 2 active areas 190a and 190b (similar to active areas 190 of FIG. 3 and FIG. 4) and the dead zone 488. For simplicity, other parts of the detector module 490 such as perimeter zones 195 (FIG. 4) are not shown. In an embodiment, a cardboard box 510 enclosing a metal sword 512 may be positioned between the detector module 490 and a radiation source (not shown) which is before the page. The cardboard box 510 is between the detector module 490 and the eye of viewer. Hereafter, for generalization, the cardboard box 510 enclosing the metal sword 512 may be referred to as the object or scene 510+512.

In an embodiment, the operation of the detector module 490 in capturing images of the object/scene 510+512 may be as follows. Firstly, the object/scene 510+512 may be stationary, and the detector module 490 may be moved to a first image capture position relative to the object/scene 510+512 as shown in FIG. 5A. Then, the detector module 490 (specifically, the active areas 190a and 190b) may be used to capture a partial image 520.1 of the object/scene 510+512 while the detector module 490 is at the first image capture position.

Next, in an embodiment, the detector module 490 may be moved to a second image capture position relative to the object/scene 510+512 as shown in FIG. 5B. Then, the detector module 490 (specifically, the active areas 190a and 190b) may be used to capture a partial image 520.2 of the object/scene 510+512 while the detector module 490 is at the second image capture position.

Next, in an embodiment, the detector module 490 may be moved to a third image capture position relative to the object/scene 510+512 as shown in FIG. 5C. Then, the detector module 490 (specifically, the active areas 190a and 190b) may be used to capture a partial image 520.3 of the object/scene 510+512 while the detector module 490 is at the third image capture position.

In an embodiment, the size and shape of the active areas 190a and 190b and the positions of the first, second, and third image capture positions may be such that any partial image of the partial images 520.1, 520.2, and 520.3 overlaps at least another partial image of the partial images 520.1, 520.2, and 520.3. For example, a distance 492 between the first and second image capture positions may be close to and less than a width 190w of the active area 190a; as a result, the partial image 520.1 overlaps the partial image 520.2.

With any partial image of the partial images 520.1, 520.2, and 520.3 overlapping at least another partial image of the partial images 520.1, 520.2, and 520.3, it is possible to stitch the partial images 520.1, 520.2, and 520.3 to form a single image 520 (FIG. 5D) of the object/scene 510+512. In an embodiment, the partial images 520.1, 520.2, and 520.3 may be stitched to form the single image 520 (FIG. 5D) of the object/scene 510+512.

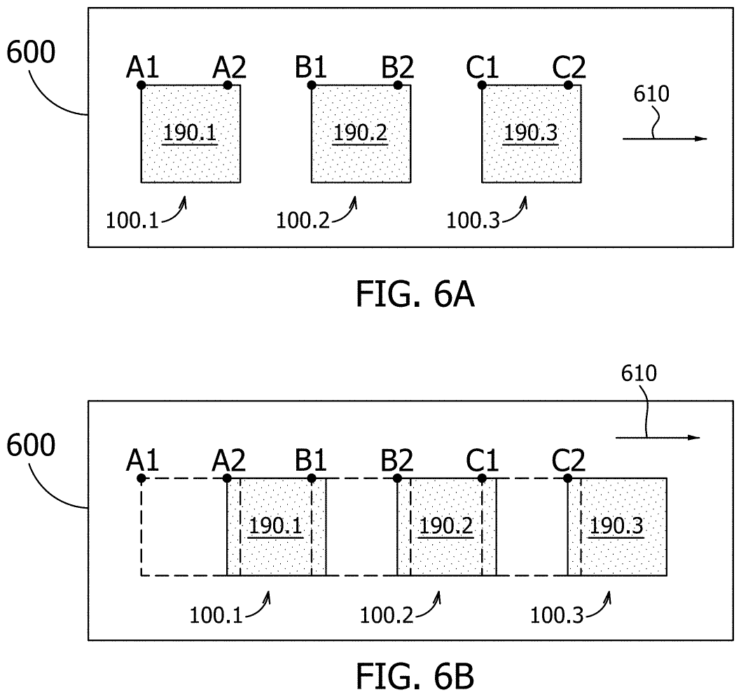

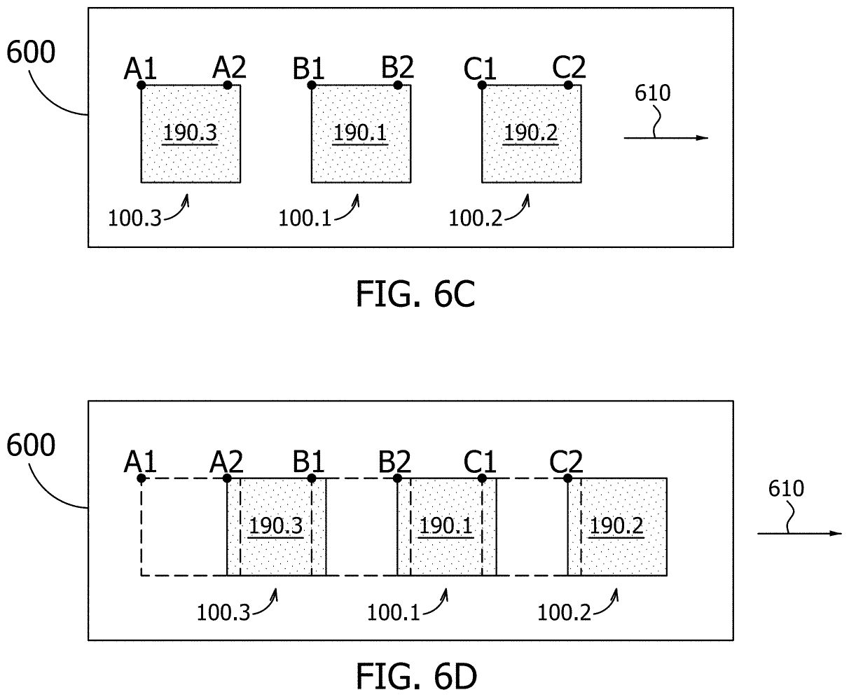

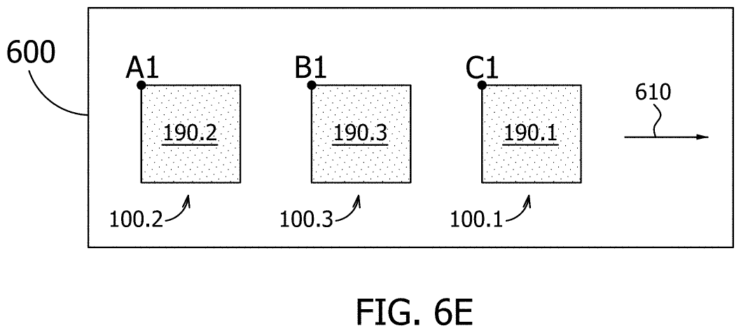

FIG. 6A-FIG. 6E schematically illustrate an operation of an imaging system 600, according to an embodiment. In an embodiment, the imaging system 600 may comprise 3 radiation detectors 100.1, 100.2, and 100.3 (or 100.1-3 for short) each of which may be similar to the radiation detector 100. For simplicity, only active areas 190.1, 190.2, and 190.3 (or 190.1-3 for short) of the radiation detectors 100.1, 100.2, and 100.3 respectively are shown.

In an embodiment, the operation of the imaging system 600 may start with a first scan of the scene by the imaging system 600 as follows. Firstly, while the top left corners of the active areas 190.1, 190.2, and 190.3 are at points A1, B1, and C1, respectively as shown in FIG. 6A, the active areas 190.1-3 may capture a first partial image of the scene.

Next, in an embodiment, the radiation detectors 100.1-3 may be moved in a scanning direction 610 such that the top left corners of the active areas 190.1, 190.2, and 190.3 are at points A2, B2, and C2, respectively. As a result of the move, all the active areas 190.1-3 are moved to the right. The result of the move is shown in FIG. 6B. In FIG. 6B, the dashed lines indicate the positions of the active areas 190.1-3 before the move. Next, in an embodiment, while the top left corners of the active areas 190.1, 190.2, and 190.3 are at the points A2, B2, and C2, respectively as shown in FIG. 6B, the active areas 190.1-3 may capture a second partial image of the scene, thereby completing the first scan of the scene by the imaging system 600.

Next, in an embodiment, a first reset of the imaging system 600 may be performed as follows. Specifically, the radiation detectors 100.1-3 may be moved such that the top left corners of the active areas 190.1, 190.2, and 190.3 are at points B1, C1, and A1, respectively. As a result of the move, the radiation detectors 100.1 and 100.2 are moved to the right, but the radiation detector 100.3 is moved from the front of the line of the radiation detectors 100.1-3 to the end of the line (i.e., to the left). The result of the move is shown in FIG. 6C.

Next, in an embodiment, the operation of the imaging system 600 may continue with a second scan of the scene by the imaging system 600. In an embodiment, the second scan may be similar to the first scan. Specifically, firstly, while the top left corners of the active areas 190.1, 190.2, and 190.3 are at points B1, C1, and A1, respectively as shown in FIG. 6C, the active areas 190.1-3 may capture a third partial image of the scene.

Next, in an embodiment, the radiation detectors 100.1-3 may be moved in the scanning direction 610 such that the top left corners of the active areas 190.1, 190.2, and 190.3 are at points B2, C2, and A2 respectively. As a result of the move, all the active areas 190.1-3 are moved to the right. The result of the move is shown in FIG. 6D. In FIG. 6D, the dashed lines indicate the positions of the active areas 190.1-3 before the move. Next, in an embodiment, while the top left corners of the active areas 190.1, 190.2, and 190.3 are at the points B2, C2, and A2, respectively as shown in FIG. 6D, the active areas 190.1-3 may capture a fourth partial image of the scene, thereby completing the second scan of the scene by the imaging system 600.

Next, in an embodiment, a second reset of the imaging system 600 may be performed. In an embodiment, the second reset may be similar to the first reset. Specifically, the radiation detectors 100.1-3 may be moved such that the top left corners of the active areas 190.1, 190.2, and 190.3 are at points C1, A1, and B1 respectively. As a result of the move, the radiation detectors 100.3 and 100.1 are moved to the right, but the radiation detector 100.2 is moved from the front of the line of the radiation detectors 100.1-3 to the end of the line (i.e., to the left). The result of the move is shown in FIG. 6E.

Next, in an embodiment, more scans and resets similar to the first scan and the first reset may be performed to get more partial images of the scene. For example, a third scan may be performed with the radiation detectors 100.1-3 in the order as shown in FIG. 6E (i.e., in the order of the radiation detectors 100.2, 100.3, and 100.1 in the scanning direction 610). After the third scan, a third reset may be performed resulting in the radiation detectors 100.1-3 physically arranged in the order of radiation detectors 100.1, 100.2, and 100.3 in the scanning direction 610 (as shown in FIG. 6A). In essence, as a result of the third reset, the radiation detector 100.1 is moved from the front of the line of the radiation detectors 100.1-3 to the end of the line.

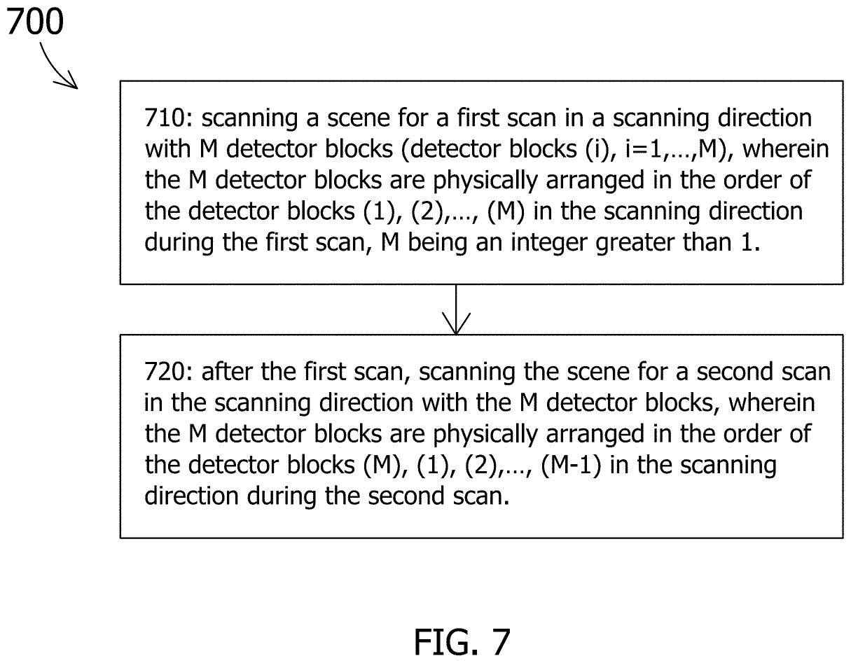

FIG. 7 shows a flowchart 700 summarizing and generalizing an operation of the imaging system 600, according to an embodiment. In step 710, M detector blocks (detector blocks (i), i=1, . . . , M) may be used to scan a scene for a first scan in a scanning direction, wherein the M detector blocks are physically arranged in the order of the detector blocks (1), (2), . . . , (M) in the scanning direction during the first scan, M being an integer greater than 1.

For example, with reference to FIG. 6A-FIG. 6B, each detector block of the M detector blocks may comprise a radiation detector 100. The 3 radiation detectors 100.1-3 (i.e., M=3) are used in the first scan in the scanning direction 610, wherein the 3 radiation detectors 100.1-3 are physically arranged in the order of the radiation detectors 100.1, 100.2, and 100.3 in the scanning direction 610 during the first scan.

In step 720, after the first scan, the M detector blocks may be used to scan the scene for a second scan in the scanning direction, wherein the M detector blocks are physically arranged in the order of the detector blocks (M), (1), (2), . . . , (M−1) in the scanning direction during the second scan. In the example above, with reference to FIG. 6C-FIG. 6D, after the first scan, the 3 radiation detectors 100.1-3 are used in the second scan in the scanning direction 610, wherein the 3 radiation detectors 100.1-3 are physically arranged in the order of the radiation detectors 100.3, 100.1, and 100.2 in the scanning direction 610 during the second scan.

In an embodiment, with reference to FIG. 6A-FIG. 6E, during each scan (e.g., the first scan, the second scan, etc.), the 3 radiation detectors 100.1-3 may be stationary with respect to each other. As a result, the lengths of the 3 straight line segments A1-A2, B1-B2, and C1-C2 are the same. In general, with reference to FIG. 7, in an embodiment, during each scan, the M detector blocks may be stationary with respect to each other.

In an embodiment, with reference to FIG. 6A-FIG. 6E, during each scan (e.g., the first scan, the second scan, etc.), the 3 radiation detectors 100.1-3 may be distributed evenly in the scanning direction 610. As a result, the lengths of the 2 straight line segments A1-B1 and B1-C1 are the same, and the lengths of the 2 straight line segments A2-B2 and B2-C2 are the same. In general, with reference to FIG. 7, in an embodiment, during each scan, the M detector blocks may be distributed evenly in the scanning direction.

In the embodiments described above, in the first scan (FIG. 6A-FIG. 6B), the active areas 190.1-3 capture the first and second partial images while the radiation detectors 100.1-3 are stationary (i.e., not moving). Similarly, in the second scan (FIG. 6C-FIG. 6D), the active areas 190.1-3 capture the third and fourth partial images while the radiation detectors 100.1-3 are stationary (i.e., not moving).

In an alternative embodiment, the active areas 190.1-3 may capture these partial images while the radiation detectors 100.1-3 are moving. For an example of this alternative embodiment, with reference to FIG. 6B, the active areas 190.1-3 may capture the second partial image while the top left corners of the active areas 190.1, 190.2, and 190.3 are moving past through the points A2, B2, and C2, respectively.

Similarly, for another example of this alternative embodiment, with reference to FIG. 6C, the active areas 190.1-3 may capture the third partial image while the top left corners of the active areas 190.3, 190.1, and 190.2 are moving past through the points A1, B1, and C1, respectively. In general, with reference to the flowchart 700 of FIG. 7, in an embodiment, for each scan, the M detector blocks may capture H partial images (H=2 in the examples above) while the M detector blocks are moving.

In an embodiment, with reference to FIG. 6A-FIG. 6E, for each scan (e.g., the first scan, the second scan, etc.), the 2 captured partial images may be stitchable together. Multiple images of a scene are stitchable together if and only if for any 2 points A and B of the scene whose images are on the multiple images, there exists a line connecting A and B such that each and every point of the line has its image on the multiple images. For example, the first and second partial images may be stitchable together. For another example, the third and fourth partial images may be stitchable together. In general, with reference to the flowchart 700 of FIG. 7, in an embodiment, for each scan, the H partial images captured by the M detector blocks may be stitchable together.

In an embodiment, with reference to FIG. 6A-FIG. 6E, for each scan (e.g., the first scan, the second scan, etc.), the 2 captured partial images may be stitched by the imaging system 600 to form an image. For example, the first and second partial images may be stitched to form an image. Another example, the third and fourth partial images may be stitched to form an image. In general, with reference to the flowchart 700 of FIG. 7, in an embodiment, for each scan, the H partial images captured by the M detector blocks may be stitched to form an image.

In an embodiment, with reference to FIG. 6A-FIG. 6E, during the first reset which occurs after the first scan (FIG. 6A-FIG. 6B) and before the second scan (FIG. 6C-FIG. 6D), the radiation detector 100.3 may be moved from the front of the line of the radiation detectors 100.1-3 to the end of the line along a path, wherein at a time point during the first reset, a point on the path is in shadows of the other radiation detectors 100.1 and 100.2 with respect to radiation used for the scans. In an embodiment, during the first reset, the radiation detector 100.3 may flip twice while it is moving along the path.

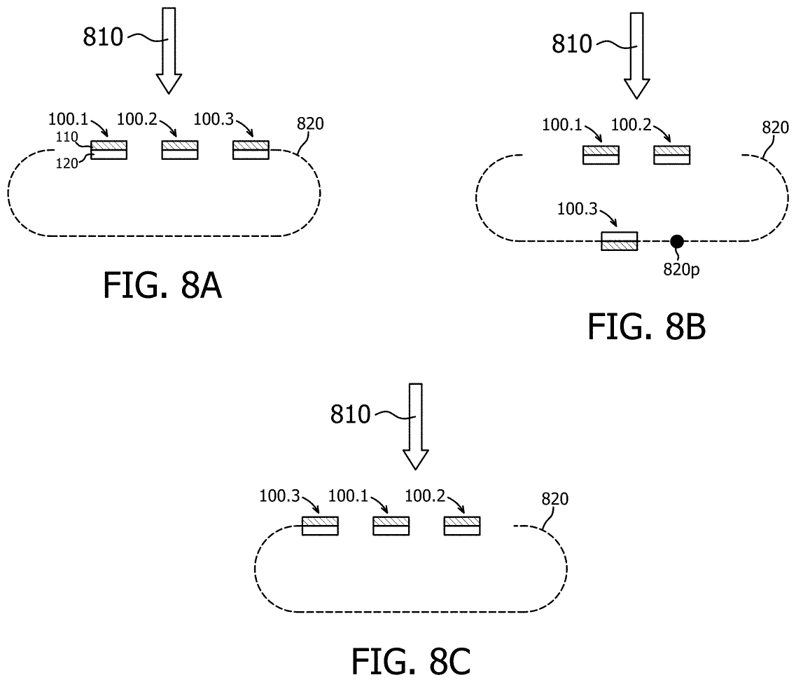

Specifically, with reference to FIG. 8A which is a side view of FIG. 6B, in an embodiment, at the end of the first scan, all the radiation absorption layers 110 of the radiation detectors 100.1-3 may face a radiation 810 used for scanning. In other words, particles of the radiation 810 hit the radiation absorption layers 110 of the radiation detectors 100.1-3 before hitting the electronics layers 120 of the radiation detectors 100.1-3.

Next, in an embodiment, during the first reset which is after the first scan and before the second scan, the radiation detectors 100.1 and 100.2 may move to the right, and the radiation detector 100.3 may move from the front of the line of radiation detectors 100.1-3 to the end of the line along a path 820. In an embodiment, with reference to FIG. 8B, at a time point during the first reset, a point 820p on the path 820 may be in shadows of the radiation detectors 100.1 and 100.2 with respect to the radiation 810.

In an embodiment, during the first reset, while moving from the front of the line of radiation detectors 100.1-3 to the end of the line, the radiation detector 100.3 may flip (i.e., its electronics layer 120 faces the radiation 810), as shown in FIG. 8B. In an embodiment, during the first reset, the radiation detector 100.3 may flip again such that at the start of the second scan as shown in FIG. 8C (which is a side view of FIG. 6C), all the radiation absorption layers 110 of the radiation detectors 100.1-3 face the radiation 810. In other words, the radiation detector 100.3 flips twice during the first reset. Such double flip movement is similar to the movement of a step of a moving walkway which is usually used in an airport.

In the embodiments described above, with reference to FIG. 7, each of the M detector blocks comprises a radiation detector 100. Alternatively, each of the M detector blocks may comprise multiple radiation detectors 100.

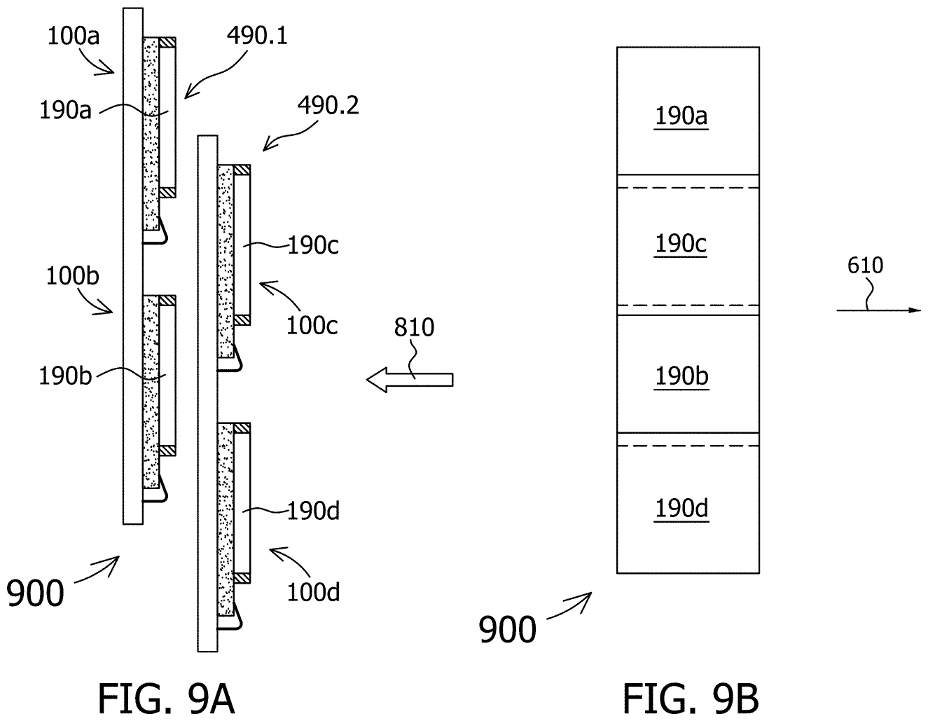

FIG. 9A schematically shows a detector block 900, according to an embodiment. For example, the detector block 900 may comprise 4 radiation detectors 100a, 100b, 100c, and 100d (or 100a-d for short) arranged on 2 detector modules 490.1 and 490.2 which may be similar to the detector module 490 (FIG. 4). In an embodiment, the 4 radiation detectors 100a-d may be stationary with respect to each other. In an embodiment, the 2 detector modules 490.1 and 490.2 may be formed on 2 separate substrates which may be bonded together to form the detector block 900.

In an embodiment, the projections of active areas 190a, 190b, 190c, and 190d of the respective radiation detectors 100a, 100b, 100c, and 100d of the detector block 900 on a plane perpendicular to the radiation 810 used for scanning collectively form a single region on the plane. In FIG. 9B, a view of FIG. 9A in the direction of the radiation 810, the plane may be the page, and the projections of active areas 190a, 190b, 190c, and 190d on the page form a single region as shown in FIG. 9B. This single region may be considered an effective active area of the detector block 900 which can detect incident radiation.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method, comprising:
scanning a scene for a first scan in a scanning direction with M detector blocks (detector blocks (i), i=1, . . . , M), wherein the M detector blocks are physically arranged in the order of the detector blocks (1), (2), . . . , (M) in the scanning direction during the first scan, M being an integer greater than 1; and
after the first scan, scanning the scene for a second scan in the scanning direction with the M detector blocks, wherein the M detector blocks are physically arranged in the order of the detector blocks (M), (1), (2), . . . , (M−1) in the scanning direction during the second scan.

2. The method of claim 1, further comprising, after the second scan, scanning the scene for a third scan in the scanning direction with the M detector blocks,
wherein the M detector blocks are physically arranged in the order of the detector blocks (M−1), (M), (1), (2), . . . , (M−2) in the scanning direction during the third scan, and wherein M>2.

3. The method of claim 1, wherein each detector block of the M detector blocks comprises a radiation detector.

4. The method of claim 1,
wherein during each scan of the first scan and the second scan, the M detector blocks are stationary with respect to each other.

5. The method of claim 4,
wherein during each scan of the first scan and the second scan, the M detector blocks are distributed evenly in the scanning direction.

6. The method of claim 1,
wherein said scanning for the first scan comprises capturing first H partial images while the M detector blocks are moving, H being an integer greater than 1, and
wherein said scanning for the second scan comprises capturing second H partial images while the M detector blocks are moving.

7. The method of claim 6,
wherein the first H partial images are stitchable together, and
wherein the second H partial images are stitchable together.

8. The method of claim 7, further comprising:
stitching the first H partial images to form an image; and
stitching the second H partial images to form an image.

9. The method of claim 1, further comprising, after the first scan and before the second scan, moving the detector block (M) along a path,
wherein at a time point after the first scan and before the second scan, a point on the path is in shadows of the other detector blocks of the M detector blocks with respect to radiation used for said first scan and said second scan.

10. The method of claim 9, wherein the detector block (M) flips twice while being moved along the path after the first scan and before the second scan.

11. The method of claim 1,
wherein each detector block of the M detector blocks comprises multiple radiation detectors,
wherein the multiple radiation detectors of said each detector block are stationary with respect to each other, and
wherein projections of active areas of the multiple radiation detectors of said each detector block on a plane perpendicular to radiation used in the first and second scans collectively form a single region on the plane.

12. An imaging system, comprising M detector blocks (detector blocks (i), i=1, . . . , M), with M being an integer greater than 1,
wherein the M detector blocks are configured to scan a scene for a first scan in a scanning direction, wherein the M detector blocks are physically arranged in the order of the detector blocks (1), (2), . . . , (M) in the scanning direction during the first scan, and
wherein the M detector blocks are configured to scan the scene for a second scan after the first scan, in the scanning direction, wherein the M detector blocks are physically arranged in the order of the detector blocks (M), (1), (2), . . . , (M−1) in the scanning direction during the second scan.

13. The imaging system of claim 12,
wherein the M detector blocks are configured to scan the scene for a third scan after the second scan, in the scanning direction, wherein the M detector blocks are physically arranged in the order of the detector blocks (M−1), (M), (1), (2), . . . , (M−2) in the scanning direction during the third scan, and wherein M>2.

14. The imaging system of claim 12, wherein each detector block of the M detector blocks comprises a radiation detector.

15. The imaging system of claim 12,
wherein during each scan of the first scan and the second scan, the M detector blocks are stationary with respect to each other.

16. The imaging system of claim 15,
wherein during each scan of the first scan and the second scan, the M detector blocks are distributed evenly in the scanning direction.

17. The imaging system of claim 12,
wherein during the first scan, the M detector blocks are configured to capture first H partial images while the M detector blocks are moving, H being an integer greater than 1, and
wherein during the second scan, the M detector blocks are configured to capture second H partial images while the M detector blocks are moving.

18. The imaging system of claim 17,
wherein the first H partial images are stitchable together, and
wherein the second H partial images are stitchable together.

19. The imaging system of claim 18,
wherein the imaging system is configured to stitch the first H partial images to form an image, and
wherein the imaging system is configured to stitch the second H partial images to form an image.

20. The imaging system of claim 12,
wherein, after the first scan and before the second scan, the imaging system is configured to move the detector block (M) along a path,
wherein at a time point after the first scan and before the second scan, a point on the path is in shadows of the other detector blocks of the M detector blocks with respect to radiation used for said first scan and said second scan.

21. The imaging system of claim 20, wherein the imaging system is configured to flip the detector block (M) twice while the detector block (M) is moved along the path after the first scan and before the second scan.

22. The imaging system of claim 12,
wherein each detector block of the M detector blocks comprises multiple radiation detectors,
wherein the multiple radiation detectors of said each detector block are stationary with respect to each other, and
wherein projections of active areas of the multiple radiation detectors of said each detector block on a plane perpendicular to radiation used in the first and second scans collectively form a single region on the plane.
